(19) United States
(12) Patent Application Publication
(10) Pub. No.: US 2025/0195225 A1
(43) Pub. Date: Jun. 19, 2025

(54) ADAPTABLE HEART VALVE HOLDER ASSEMBLY

(71) Applicant: EDWARDS LIFESCIENCES CORPORATION, Irvine, CA (US)

(72) Inventors: Brian S. Conklin, Orange, CA (US); Louis A. Campbell, Santa Ana, CA (US)

(21) Appl. No.: 19/067,768

(22) Filed: Feb. 28, 2025

Related U.S. Application Data
(63) Continuation of application No. 17/244,771, filed on Apr. 29, 2021, now abandoned.
Application No. 17/244,771 is a continuation of application No. PCT/US2019/058642, filed on Oct. 29, 2019.
(60) Provisional application No. 62/754,070, filed on Nov. 1, 2018.

(51) Int. Cl. A61F 2/24; A61F 2/95

(52) U.S. Cl. CPC A61F 2/2466; A61F 2/2445; A61F 2/2496; A61F 2002/9505; A61F 2/9517; A61F 2220/0075

(57) ABSTRACT

An annuloplasty ring assembly with detachable handle includes an annuloplasty ring configured to be secured to an annulus of a heart valve and an annuloplasty ring. The annuloplasty ring is attached to the ring holder such that the annuloplasty ring is in conformal contact along a lower surface of a peripheral edge portion of the ring holder. The assembly includes a handle adapter extending proximally from an upper proximal face of the ring holder. The handle adapter is also attached or attachable to a distal attachment end of the handle on one side and removably attached to the annuloplasty ring holder on another side.

### CROSS REFERENCE TO RELATED APPLICATIONS

This application is a continuation of U.S. application Ser. No. 17/244,771, filed Apr. 29, 2021, which is a continuation of International Patent Application No. PCT/US2019/058642, filed Oct. 29, 2019, which claims the benefit of U.S. Patent Application No. 62/754,070, filed Nov. 1, 2018, the entire disclosures all of which are incorporated by reference for all purposes.

This application is related to U.S. Patent Application No. 62/754,091, filed Nov. 1, 2018, and to U.S. Patent Application No. 62/754,066, filed Nov. 1, 2018, the entire disclosures of both applications incorporated by reference herein for all purposes.

### TECHNICAL FIELD

The present disclosure relates generally to medical devices and to tools for delivering such medical devices. More specifically, the disclosure relates to the surgical repair of native heart valves that have malformations and/or dysfunctions. Embodiments of the invention relate to detachable handles for facilitating the repair of heart valves with annuloplasty rings and methods of implanting annuloplasty rings using these handles.

### BACKGROUND

Referring to FIG. 1, the human heart is generally separated into four pumping chambers which pump blood through the body. Each chamber is provided with its own one-way exit valve. The left atrium receives oxygenated blood from the lungs and advances the oxygenated blood to the left ventricle through the mitral (or bicuspid) valve. The left ventricle collects the oxygenated blood from the left atrium and pushes it through the aortic valve to the aorta, where the oxygenated blood is then distributed to the rest of the body. Deoxygenated blood from the body is then collected at the right atrium and advanced to the right ventricle through the tricuspid valve. The right ventricle then advances the deoxygenated blood through the pulmonary valve and the pulmonary artery to the lungs to again supply the blood with oxygen.

Each of the valves associated with the chambers of the heart are one-way valves that have leaflets to control the directional flow of the blood through the heart, and to prevent backflow of the blood into other chambers or blood vessels that are upstream of the particular chamber. The valves are each supported by an annulus having a dense fibrous ring attached either directly or indirectly to the atrial or ventricular muscle fibers.

When a valve becomes diseased or damaged, the efficiency and/or general functionality of the heart may be compromised. Diseased heart valves may be categorized as either stenotic, wherein the valve does not open sufficiently to allow adequate forward flow of blood through the valve, and/or incompetent, wherein the valve does not close completely, causing excessive backward flow of blood through the valve when the valve is closed. Valve disease can be severely debilitating and even fatal if left untreated.

Various surgical techniques may be used to repair a diseased or damaged valve. One repair technique that has been shown to be effective in treating incompetence is annuloplasty, in which the effective size and/or shape of the valve annulus is modified by securing a repair segment, such as an annuloplasty ring, around all or a portion of the heart valve annulus. For example, the valve annulus may be contracted by attaching a prosthetic annuloplasty ring to an interior wall of the heart around the valve annulus. The annuloplasty ring is designed to support the functional changes that occur during the cardiac cycle, maintaining coaptation and valve integrity to prevent reverse flow while permitting good hemodynamics during forward flow.

The annuloplasty ring typically comprises an inner substrate, often formed from a metal (such as stainless steel, titanium, or Nitinol) or from a flexible material (such as silicone rubber or Dacron cordage), which is typically covered with a biocompatible fabric or cloth, forming a sewing cuff, to allow the ring to be sutured to the heart tissue. Depending on a particular application, annuloplasty rings may be stiff or flexible, may be split (including rings that extend at least halfway around the valve annulus) or continuous, and may have a variety of shapes, including circular, D-shaped, C-shaped, saddle-shaped, and/or kidney-shaped. Examples are seen in U.S. Pat. Nos. 5,041,130, 5,104,407, 5,201,880, 5,258,021, 5,607,471, 6,187,040, and 6,805,710, the contents of which are all incorporated herein by reference in their entirety. Many annuloplasty rings are formed in a plane, but some rings are generally non-planar. Such non-planar rings can be saddle-shaped, and/or bowed along various portions, such as being bowed along their anterior or straight side to conform to the desired shape of the annulus at that location.

In the usual mitral annuloplasty ring implant procedure, an array of separate implant sutures are first looped through all or portions of the exposed mitral annulus at intervals spaced equidistant from one another, such as, for example, 4-mm intervals. The surgeon then threads the implant sutures through the annuloplasty ring at more closely spaced intervals, such as, for example, 2-mm. This occurs with the prosthesis outside the body, typically secured to a peripheral edge portion of a holder or template. The ring on the holder is then advanced (parachuted) distally along the array of pre-anchored implant sutures into contact with the valve annulus, thus effecting a reduction in valve annulus circumference. At this point a handle used to manipulate the holder or template may be detached for greater visibility of the surgical field. The surgeon ties off the implant sutures on the proximal side of the ring, and releases the ring from the holder or template, typically by severing connecting sutures at a series of cutting guides. Examples of annuloplasty ring implant procedures are described in U.S. Pat. Nos. 8,216,304 and 8,152,844, the contents of which are all incorporated herein by reference in their entirety.

Current mitral repair rings on the market have holder systems that utilize a snap-in feature to lock their handles into the holders. While the use of snap-in features may be convenient for initially attaching the handles to the holders, it may be difficult to separate the handle from the holder during implantation, especially if the prosthesis is already parachuted to the annulus. With the current systems, the handle is generally removed along with the holder itself during implantation. It may be desirable, however, to leave the holder in place until all the knots are tied in some cases, while removing the handle for better access/less obstruction. This could be particularly important with flexible bands and rings, as it could be beneficial to leave the holder attached to the prosthesis to define its shape while tying it down to the annulus. Likewise, for minimally invasive surgical (MIS) procedures, it may be desirable to have a handle attached to the implant while placing sutures in it and then be able to remove the handle while leaving the holder attached and parachute the implant using an MIS instrument.

Some current holders for mitral repair rings have a snap-fit mechanism located on the end of a long (˜5 cm: ˜2 inch) stem to attach a handle to the implant holder. Even with the handle removed, there is still a stem that is nearly 5-cm (about 2-inches) long attached to the holder that cannot be removed, hindering access to the entire circumference of the ring during implantation. One other concern is that many prior holders are attached at a pre-determined angle relative to the implant. The angle may be well suited to one surgical approach, such as a full sternotomy, but poorly suited to a procedure such as a right thoracotomy.

Given the above, it would be desirable to have a mitral repair implant holder that allows the handle to be attached and then quickly and easily detached at any time during the procedure, independent of the holder. Removing the handle allows for better access to the prosthesis for knot tying and for better compatibility with minimally invasive surgical procedures. It would also be desirable to have a minimum holder profile, instead of a two-inch stem, once the handle is removed. It would also be desirable to have a holder system for a mitral ring that would have options for attaching the handle at multiple locations which would be amenable to different surgical approaches (such as a sternotomy or a thoracotomy).

Current holder systems also include a reusable sizer set and tray to account for proper sizing of the native valve annulus. Accordingly, the surgeon is often provided with a tray of sizer heads having perimeter shapes and sizes similar to those of the annuloplasty rings to be implanted. The one that best fits the native valve annulus is used to select the proper annuloplasty ring. Present methods of sizing a valve annulus, however, are inefficient and awkward to employ due to the need to use additional instruments during the sizing procedure. This is especially true for procedures to size an annulus or deliver an implant through a minimal size incision. In addition, any changes to the sizers or tray would necessitate many validations. The sizer and handle sets must be sterilized and sent to the operating room in advance of the procedure, which leaves room for error.

In view of the above, it would be desirable to have a modular system that uses the same handle for both sizing and ring implantation. This would give the surgeon an efficient process for both sizing and implanting the annuloplasty ring. It would also be desirable to have a ready to use holder and sizer system for a mitral ring or band that could use a disposable handle for both sizing and implantation. The sizer heads could also be disposable to avoid costly packaging validation associated with a new reusable tray of sizer heads.

### SUMMARY

In one embodiment of the present invention, an annuloplasty ring assembly with detachable handle includes an annuloplasty ring configured to be secured to an annulus of a heart valve and an annuloplasty ring holder. The annuloplasty ring is removably attached to the ring holder and is in conformal contact along a lower surface of a peripheral edge portion of the ring holder. A handle adapter extends proximally from an upper proximal face of the ring holder and the handle adapter is attached or attachable to a distal attachment end of the handle on one side and removably attached to the annuloplasty ring holder on another side. In a preferred embodiment, the upper proximal face of the ring holder forms an opening and the handle adapter is located in the opening of the ring holder. In addition, the handle adapter is removably attached to the ring holder by a suture.

In one embodiment, the handle adapter is overmolded to the distal attachment end of the handle. In another embodiment, the distal attachment end of the handle and the handle adapter are each threaded for cooperative engagement. In a further embodiment, the distal attachment end of the handle and the handle adapter form a snap in engagement to secure the handle to the handle adapter.

In another embodiment, the handle adapter has two attachment locations and the distal attachment end of the handle can be selectively attached to each attachment location. The distal attachment end of the handle and each of the two attachment locations of the handle adapter may be threaded for cooperative engagement. Alternatively, the distal attachment end of the handle and each of the two attachment locations of the handle adapter form a snap in engagement to secure the handle to the handle adapter.

In another embodiment, the annuloplasty ring assembly further includes a set of sizer heads. Each sizer head has a sizer adapter that is removably attachable to the distal attachment end of the handle. The sizer adapter of each sizer head and the distal attachment end of the handle form a snap in engagement to reversibly secure the handle to the sizer head.

In another embodiment, the handle adapter is removably attached to the ring holder by a suture and the ring holder further includes a cutting well projecting up from the upper proximal face of the ring holder. The suture is arranged across the cutting well and is accessible for cutting to release the handle adapter from the ring holder. Preferably, the ring holder has a low profile such that the cutting well is the largest projection from the upper proximal face of the ring holder.

In another embodiment, the annuloplasty ring assembly includes an annuloplasty ring, an annuloplasty ring holder, a handle with a distal attachment end, and a handle adapter extending proximally from an upper proximal face of the ring holder. The handle adapter has two attachment locations and a distal attachment end of the handle can be selectively attached to each attachment location. Preferably, the distal attachment end of the handle and each of the two attachment locations of the handle adapter are threaded for cooperative engagement.

Other embodiments provide a method for delivering and implanting an annuloplasty ring in a patient in need thereof using a holder or annuloplasty ring assembly disclosed herein.

### BRIEF DESCRIPTION OF THE DRAWINGS

Further features and advantages of the invention will become apparent from the description of embodiments using the accompanying drawings. In the drawings:

FIG. 1 is a schematic cross-sectional view of the human heart;

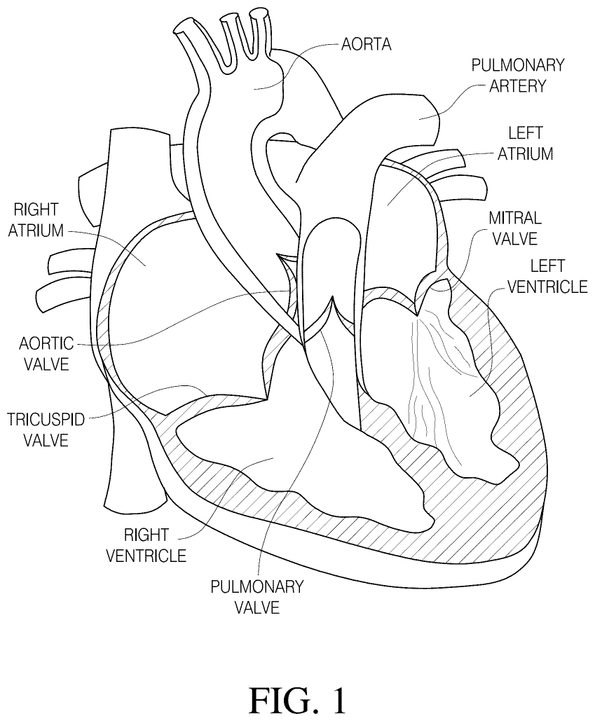

FIG. 2 is an exploded perspective view of a first embodiment of an annuloplasty ring assembly;

FIG. 3 is an assembled top perspective view of the annuloplasty ring assembly of FIG. 2;

FIG. 4 is an assembled side view of the annuloplasty ring assembly of FIG. 2;

FIG. 5 is a cross-sectional view of the ring holder of the annuloplasty ring assembly of FIG. 4;

FIG. 6 is an assembled bottom perspective view of the annuloplasty ring assembly of FIG. 2;

FIG. 7 is an exploded perspective view of a second embodiment of an annuloplasty ring assembly;

FIG. 8 is an exploded perspective view of a third embodiment of an annuloplasty ring assembly;

FIG. 9 is an assembled perspective view of the annuloplasty ring assembly of FIG. 8;

FIG. 10 is an exploded top perspective view of the handle tip of FIG. 8 and a sizer head;

FIG. 11 is an assembled top perspective view of the handle tip and sizer head of FIG. 10;

FIG. 12 is an exploded bottom perspective view of the handle tip and sizer head of FIG. 10;

FIG. 13 is an exploded perspective view of a fourth embodiment of an annuloplasty ring assembly;

FIG. 14 is an assembled perspective view of the annuloplasty ring assembly of FIG. 13 with the handle in a first attachment location;

FIG. 15 is an assembled perspective view of the annuloplasty ring assembly of FIG. 13 with the handle in a second attachment location.

### DETAILED DESCRIPTION

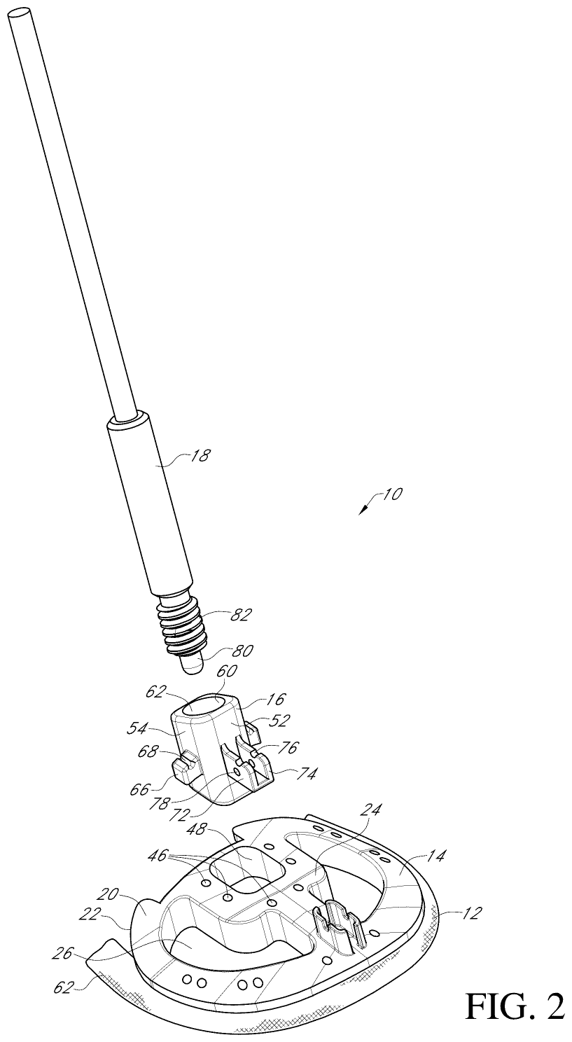

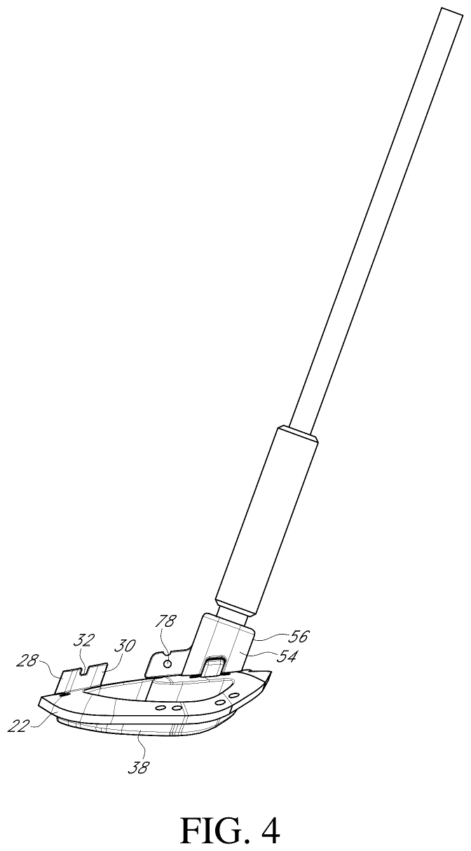

With reference to FIGS. 2-6, an embodiment of an annuloplasty ring assembly 10 includes an annuloplasty ring 12, a ring holder 14, a handle adapter 16 and a handle 18. In this embodiment, the annuloplasty ring 12 is C-shaped and open on one side. The annuloplasty ring may have a generally rigid or flexible core (not shown) surrounded by an outer cover 62 such as a layer or tube of silicone rubber covered with a woven polyester cloth.

The ring holder 14 is defined by an upper proximal face 20, an outer peripheral edge portion 22 and a crossbar 24 extending from one side of the peripheral edge portion 22 to another side. The peripheral edge portion 22 has the same shape in plan view as the annuloplasty ring that it is designed to hold. Between the peripheral edge portion 22 and the crossbar 24, the ring holder 14 provides a pair of relatively large visibility windows 26 that together occupy a large cross-sectional area within the peripheral edge portion. The windows 26 allow the surgeon to see distally through the ring holder 14 and the annuloplasty ring to evaluate the condition of the valve annulus and ventricular structures as the ring is implanted.

A cutting well 28 projects upward from the proximal face 20 of the ring holder at a narrow end of the crossbar. Walls 30 of the cutting well 28 are located adjacent the peripheral edge portion 22 and extend upward from the proximal face 20. A notch 32 is provided on an upper edge of each wall 30. The notches 32 provide a convenient bridge across which a connecting suture or sutures (not shown) are suspended. Between the walls 30 is a gap 34 to provide space for a suture cutting instrument. The walls 30 present one configuration of cutting well that may be utilized, and of course others are contemplated.

The ring holder 14 also includes a series of through holes 36 for passage of a suture for firmly holding the annuloplasty ring 12 to the ring holder 14. It should be understood that although through holes are the preferred construction, other configurations that provide passages through the ring holder and/or perform similar functions are contemplated. It will also be appreciated that sutures used to hold the annuloplasty ring and the ring holder during the implanting procedure should be attached to the ring holder to insure that those sutures are removed together with the ring holder after the annuloplasty ring is delivered to the native valve annulus. Examples of suture routing are described in U.S. Pat. No. 8,152.844, the contents of which are incorporated herein by reference in their entirety.

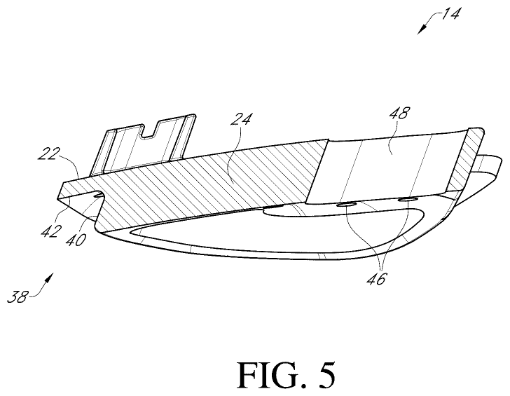

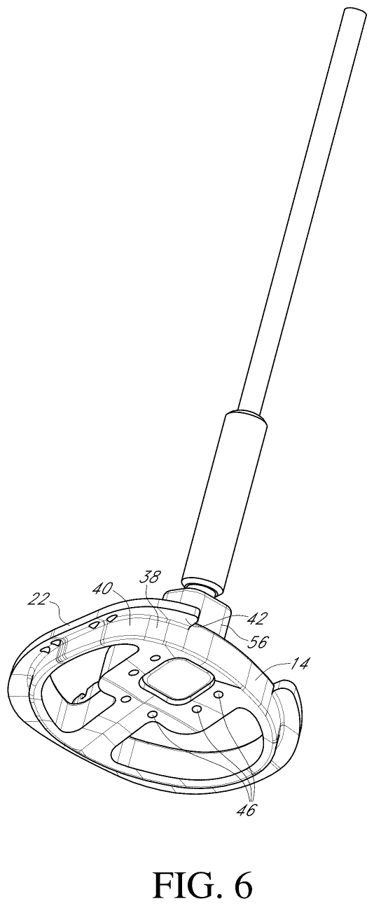

With reference to FIGS. 5 and 6, the annuloplasty ring conforms to an angled channel 38 of the ring holder 14 defined by a generally axially extending distal wall 40 and an outwardly extending proximal ledge 42 forming an outer extent of the peripheral edge portion 22. The annuloplasty ring is secured inside and around the channel and follows a three-dimensional path, and the peripheral edge portion 22 and the channel 38 mirror this three-dimensional shape.

To remove the annuloplasty ring 12 from the holder 14, a member of the surgical team will cut the retaining sutures that hold the ring 12 to the holder 14. A scalpel or other cutting instrument is passed into the gap 34 of the cutting well 28 to cut the retaining sutures. With the retaining sutures cut, the holder 14 can be pulled away from the annuloplasty ring 12. Note that in an exemplary embodiment the retaining sutures are connected via knots or other restraints to the holder 14, so that when the retaining sutures are cut their severed ends will remain connected to the holder 14. Accordingly, when the retaining sutures are cut and the holder 14 pulled away from the ring 12, the retaining sutures will be removed from the ring 12 along with the holder 14.

The width of the crossbar 24 at an end near the cutting well 28 is narrow, whereas the width of the crossbar at the opposite end is wide and defines an opening 48 for receiving the handle adapter 16. Around the opening 48 are several suture holes 46 for securing the handle adapter 16 to the ring holder 14.

The handle adapter 16 has four walls—a front wall 52, two side walls 54 and a back wall 56. A bore 60 is formed into a top surface of the handle adapter. The bore extends into the handle adapter 16 to receive the handle 18 and may be open or closed at the bottom end. The bore 60 has an upper portion 64 that is threaded and a lower portion (not shown) that is unthreaded. The lower portion of the bore has a smaller diameter than the upper portion of the bore.

A suture tab 66 extends from each side wall 54 of the handle adapter 16. Each suture tab 66 has an upper groove 68 to receive a suture 70. Projecting from the front wall is a cutting well 72 formed from two walls 74 that are spaced from each other. A top of each wall has a groove 76 for receiving a suture.

The handle 18 has a lead-in portion 80 at its distal end. Next to the lead-in portion 80 is a threaded portion 82 that has a greater diameter than the lead-in portion. The handle 18 is assembled to the handle adapter 16 by threading it into the handle adapter with a few turns. The lead-in portion 80 has a similar diameter as the lower portion of the bore 60 of the handle adapter 16 to ensure axial alignment of the threads and thereby greatly reduces the incidence of cross-threading and possible particle generation. Preferably, the handle 18 has a malleable shaft that can be stainless steel, aluminum, nitinol, or some other malleable material.

The combined handle 18 and handle adapter 16 are assembled to the ring holder 14 by inserting a distal end of the handle adapter 16 into the opening 48 of the ring holder 14. The handle adapter is inserted into the opening 48 until the suture tabs 66 and the cutting well 72 stop against the proximal face 20 of the ring holder 14.

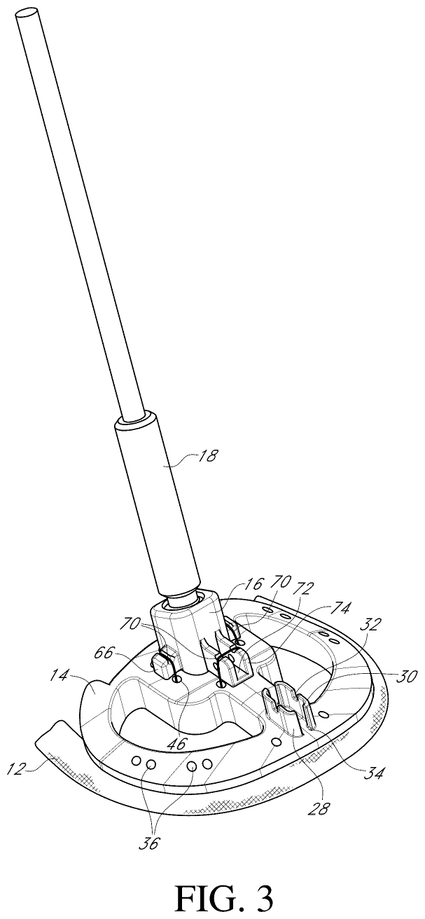

With reference to FIG. 3, the suture 70 holds the handle adapter 16 to the ring holder 14. One end of the suture 70 is secured to one wall 74 of the cutting well 72 of the handle adapter 16, for example, by threading the suture through a hole or aperture 78 in the wall 74 and tying a knot to secure the suture to the wall. The suture may then be passed over the cutting well 72 and placed in the grooves 76 of the walls 74. The suture is then passed down along the adjacent wall 74 of the cutting well 72 and routed through the suture hole 46 in the ring holder 14. The suture 70 then passes along the bottom of the ring holder to a location below the front of the suture tab 66 in the side wall 54 of the handle adapter 16. The suture is then routed through the suture hole 46 in the ring holder and up and over the suture tab 66. Then back down through the ring holder and across to the other side of the handle adapter where the suture is similarly threaded through a suture hole, over the opposite suture tab, down through the ring holder and back up to the cutting well where it is tied off. Alternatively, the suture may be secured at the cutting well 72 only, without passing over the suture tabs 66, e.g., by passing down through hole 46 adjacent one wall 74, then under the cutting well 72 and back up through the ring holder and adjacent the other wall 74 to be tied off.

Cutting the single suture 70 allows the handle 18 and threaded adapter 16 to be released from the ring holder 14 by pulling them away from the ring holder. The suture 70 stays attached to the adapter 16 and the annuloplasty ring remains attached to the ring holder. Removing the handle 18 allows better access to the sewing ring of the implant, thus making it easier to tic down the implant sutures. At the same time, the holder 14 remains attached to the implant, thereby helping to maintain its shape during tie-down. This could be particularly important in the case of flexible implants where it is desirable to maintain the shape of the implant until it is fully secured to the annulus.

Cutting the single suture 70 can be done after parachuting the annuloplasty ring to the valve annulus, or in a minimally invasive surgical (MIS) procedure before parachuting the valve to the annulus. With the handle and/or the adapter removed, the holder can be grasped with an MIS or robotic instrument to facilitate surgical techniques with minimal-sized incisions. As can be seen in FIG. 2, with the adapter 16 removed, the ring holder 14 has a very low profile permitting easier access for tooling and better visibility. The holder 14 can remain attached to the implant 12 until all the sutures are tied to ensure the implant stays in its desired shape during implantation. This is particularly important for flexible implants. Removing the handle and/or the adapter allows 360-degree access to the implant for knot tying and verifying placement. The ring holder 14 can then be removed from the annuloplasty ring 12 by cutting any sutures across the cutting well 28 of the holder 14. Also note that a suture guard could be incorporated into the handle adapter 16 that covers the suture cut gap 34 shown in FIG. 3 to prevent cutting that suture first before cutting the suture 70 to release the handle 18. An example of a suture guard is disclosed in U.S. Patent Application Publication No. 2018/0116795, the contents of which are all incorporated herein by reference in their entirety.

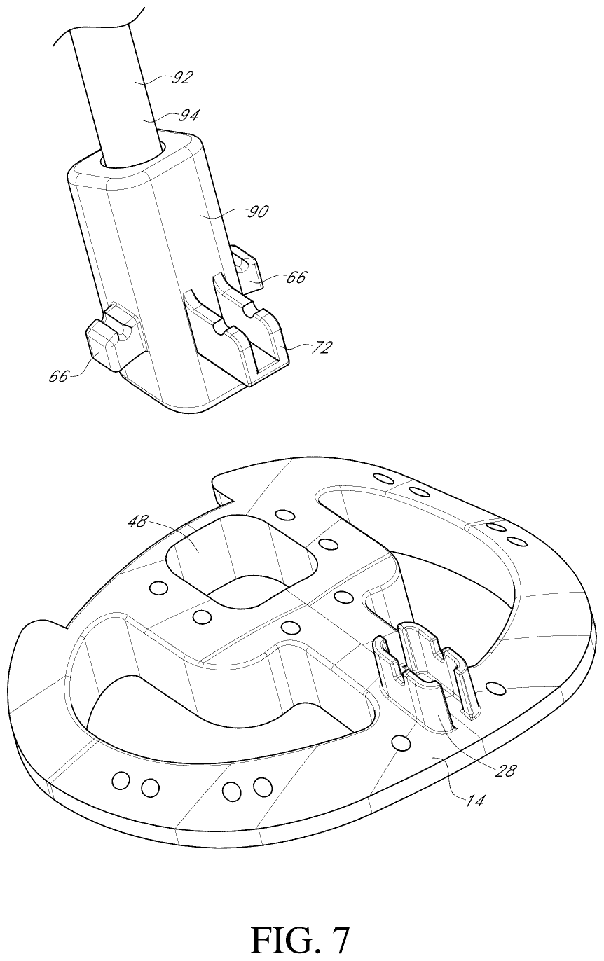

In an alternative embodiment shown in FIG. 7, the handle adapter 90 has a disposable, pre-attached handle 92 that can be easily removed from the ring holder 14 during the implant procedure. This embodiment also leaves the holder 14 attached to the annuloplasty ring 12 for implant tie-down in order to maintain its shape. In this embodiment, the length of the handle adapter 90 may be extended. In particular, the portion of the handle adapter 90 above the suture tabs 66 and cutting well 72 has a greater length than in the previous embodiment. The handle adapter 90 may then be overmolded onto a distal end of the handle 92 having a malleable shaft 94 made of a material such as stainless steel, aluminum, nitinol or other suitable material. Overmolding the components is inexpensive and avoids difficult to validate adhesive bonding steps during manufacture. As in the previous embodiment, the distal end of the adapter 90 is inserted into the opening 48 of the ring holder 14, and then attached to the ring holder 14 at the cutting well 72 by means of a single suture that can be cut to remove the handle and adapter.

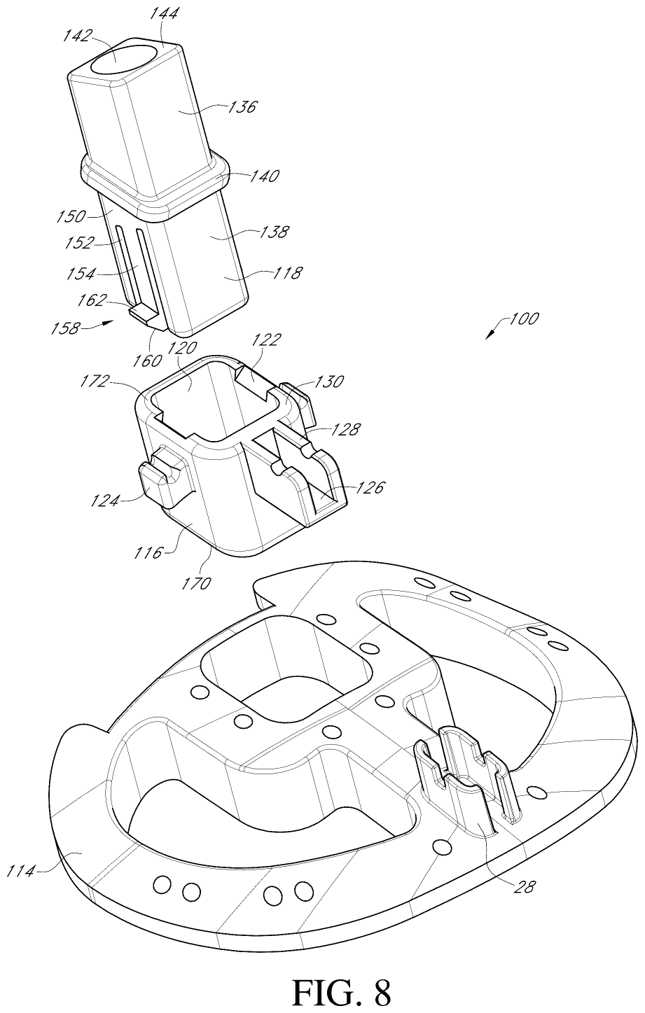

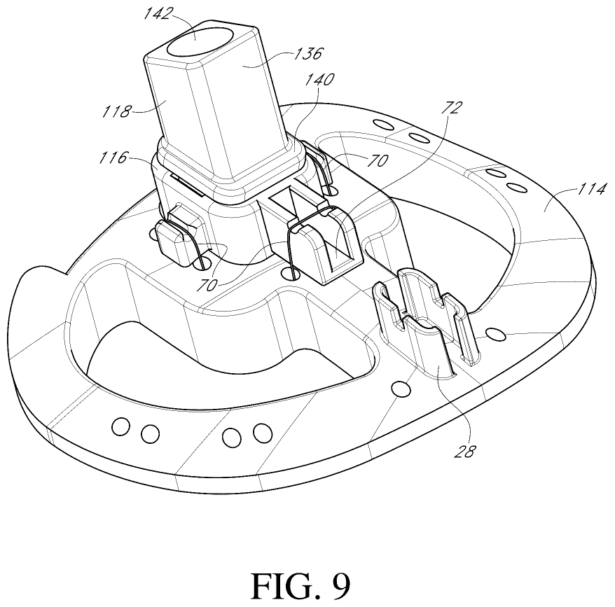

With reference to FIGS. 8-9, another embodiment of an annuloplasty ring assembly 100 includes a ring holder 114, a handle adapter 116 and a handle tip 118. The ring holder 114 is the same as in the previous embodiments. The handle adapter 116 has four walls as in previous embodiments, but in this case, has a rectangular bore 120 running through it. An upper portion of the rectangular bore defines a pair of opposed ramps 122. As in previous embodiments, the handle adapter has suture tabs 124 on the side walls and a cutting well 126 on the front wall. In this case, top surfaces 128 of the walls of the cutting well are aligned with a top surface 130 defining the rectangular bore 120.

The handle tip 118 is a rectangular member having an upper portion 136 and a bottom portion 138. A shoulder 140 midway between the ends of the rectangular member separates the upper and lower portions. A bore 142 extends into the upper portion through a top surface 144 of the handle tip 118 to receive a shaft 94 of a handle 92. The shaft 94, like the shaft in the previous embodiment, is preferably a malleable material such as stainless steel, aluminum or nitinol. The malleable shaft could be overmolded on one end to the handle tip and on the other end to a handle grip.

The bottom portion includes a solid block 150 adjacent the shoulder 140, then extends downward in a solid H-shape that forms a pair of channels 152. Located in each channel is a flexible arm 154 that extends from the solid block 150. A distal end of the flexible arm 154 has an outwardly projecting snap-in feature 158 forming a ramp 160 and a stop 162. Each flexible arm 154 is spaced from the solid H-shape to permit inward flexing sufficient to actuate the snap-in function. The stop 162 is preferably a flat surface perpendicular to the handle tip axis.

The embodiment is assembled by inserting the handle tip 118 into the rectangular bore 120 of the handle adapter 116. The ramps 160 of the flexible arms 154 engage the opposed ramps 122 of the upper portion of the bore 120 of the handle adapter 116 to flex the arms 154 inward. After the handle tip 118 passes through the bore 120, the arms 154 flex back out and the stops 162 engage a bottom surface 170 of the handle adapter 116 while the shoulder 140 engages a top surface 172 of the handle adapter 116 to secure the handle tip 118 in place. The handle adapter is secured to the ring holder by a single suture as described in previous embodiments.

Preferably, the bottom surface 170 is parallel to the flat surface of the stop 162. In this way, the snap fit feature of the handle tip 118 and the handle adapter 116 are designed to be nonreversible and the implant is not able to “pop off” the handle inadvertently due to forces associated with parachuting the ring to the annulus or adjusting its position. Instead, the handle, the handle tip 118, and the handle adapter 116 are removed from the implant holder 114 by cutting a single suture. As in the previous embodiment, the suture stays attached to the adapter. Removing the handle allows better access to the sewing ring of the implant, thus making it easier to tie down the implant sutures. At the same time, the holder 114 remains attached to the implant, thereby helping to maintain its shape during tie-down. This could be particularly important in the case of flexible implants where it is desirable to maintain the shape of the implant until it is fully secured to the annulus.

Once tie-down of the annuloplasty ring is complete, the suture is cut at the cutting well 28 of the ring holder 114 to release and remove the ring holder from the annuloplasty ring. As noted above, a suture guard could be incorporated into the handle adapter 116 that covers the suture cut location at the cutting well 28 to prevent cutting that suture first before cutting the suture 70 at 72 to release the handle.

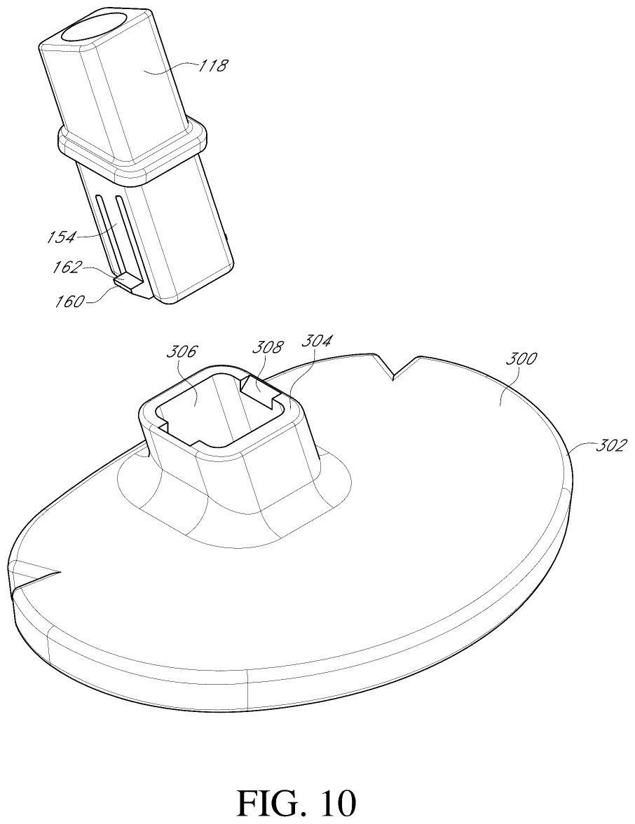

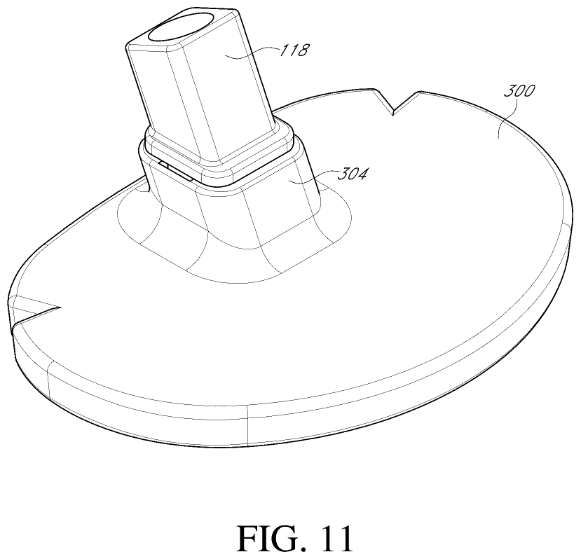

Annuloplasty rings come in many sizes and it is important for the surgeon to select the size that best fits the native valve annulus. A tray (not shown) of sizer heads may be provided for this purpose. With reference to FIGS. 10-12, a sizer head 300 has a periphery 302 that is generally shaped the same as an annuloplasty ring. For a tray of sizer heads, several sizer heads with different periphery shapes are provided. In selecting the correct sizer head 300, the surgeon selects the one that matches the valve annulus shape. Each sizer head is also provided with a sizer adapter 304 that projects from the top of the sizer head. The sizer adapter 304 has four walls and a rectangular bore 306 running through it. An upper portion of the rectangular bore 306 defines a pair of opposed ramps 308 at the top of the bore. A lower portion of the bore also has opposed recesses 310 with ramps 312. The adapter 304 may be molded together with the sizer head 300 or attached as a separate part.

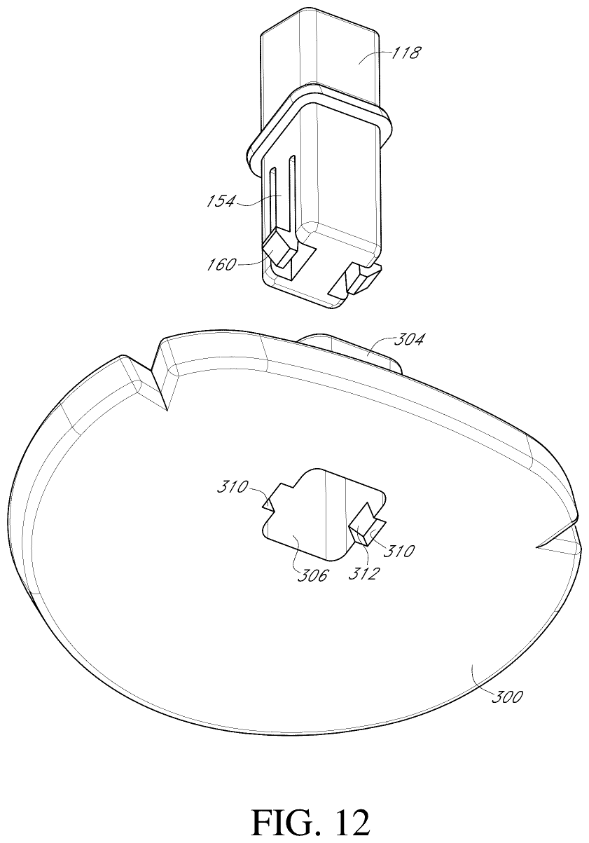

The same handle tip 118 used with the handle adapter 116 (see FIG. 8) can be used with the sizer head 300. The difference here is that the ramps 312 at the lower portion of the bore 306 form an angled release ramp 312 (see FIG. 12) to permit release of the sizer head 300.

To attach the handle tip 118 to a sizer head 300, the handle tip 118 is inserted into the bore 306 of the adapter 304. The ramps 160 of the flexible arms 154 engage the opposed ramps 308 of the bore 306 to flex the arms 154 inward. After the handle tip 118 passes through the bore 306, the arms 154 flex back out and the stops 162 engage the angled release ramps 312 of the sizer head 300 to hold the handle tip 118 and sizer head 300 together. To remove the sizer head, the handle tip 118 is pulled back in the opposite direction. The sizer heads reversibly attach to the handle tip because the release ramp 312 will hold the sizer head until sufficient force is applied to the handle tip to deflect the arms 154 past the release ramp 312. The amount of force needed to release the sizer heads from the handle tip could be optimized by changing the angle of the release ramp.

During use, the surgeon selects a first sizer head to try. A member of the surgical team then attaches that sizer head to the handle tip. If the surgeon desires to try a different size, the first sizer head is removed and the next head is attached using the same procedure. This process is repeated until the surgeon determines the correct size. Sizer heads can easily be attached and detached, thereby allowing the surgeon to try a variety of different sizer heads until the correct size is determined.

Accordingly, a sizer and implant holder system for mitral annuloplasty rings is described that comes with a set of disposable sizer heads, a disposable handle with a malleable shaft, and a compatible ring holder attached to an annuloplasty ring. The snap fit features of the handle tip and the sizer heads are designed to be a reversible fit. In this way, the surgeon can try several different sizer heads, one by one, on the handle tip by simple pulling one off and snapping another on. The handle would come with the entire range of sizer heads. The handle can also attach to the ring holder of FIG. 9 and be released with cutting a single suture. This leaves the holder attached to the implant for implant tie-down in order to maintain its shape.

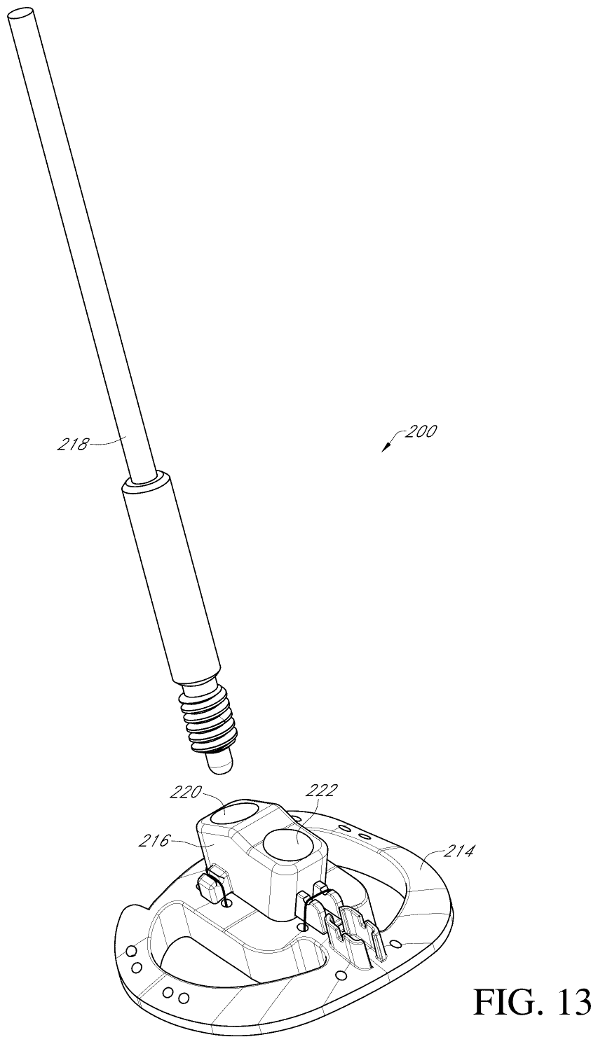

With reference to FIGS. 13-15 a further embodiment of an annuloplasty ring assembly 200 includes a ring holder 214, a handle adapter 216, and a handle 218. The ring holder 214 and the handle 218 are the same as in the first embodiment.

The handle adapter 216 projects upward from the ring holder 214 and forms a first threaded hole 220 and a second threaded hole 222. The holes have non-parallel axes. Each hole can include a lead-in section (not shown) at the bottom that helps insure axial alignment between the handle 218 and the adapter 216 and thus prevent cross-threading. Also, the threads could be made in such a way that only about 1-2 full rotations is needed to attach the handle.

The handle adapter 216 allows the handle 218 to be attached in one of two different locations, one ideally suited for a sternotomy, and the other ideally suited for a thoracotomy. These two surgical approaches have very different angulations relative to the plane of the mitral valve annulus and therefore need a holder that can address either need.

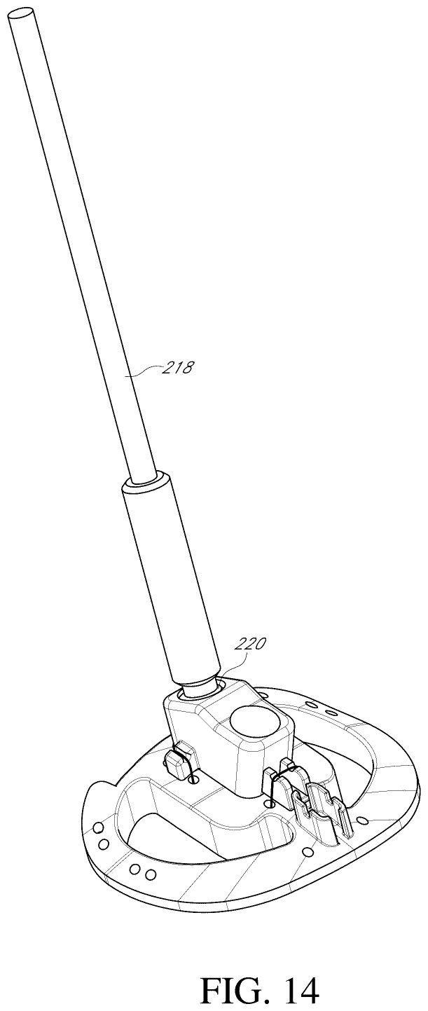

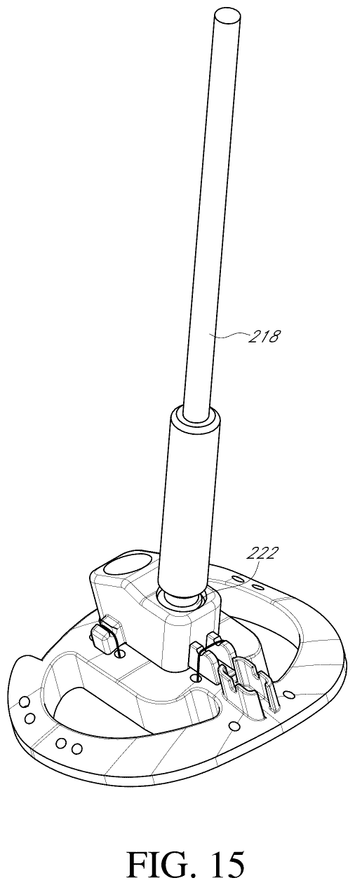

For a sternotomy, the handle is located in the first threaded hole 220 at an angle relative to the plane of the implant to help compensate for the angle between the mitral valve annulus and the entry from the sternotomy (FIG. 14). For a thoracotomy, the mitral valve annulus is generally a “straight shot” from the incision, and therefore axial alignment between the handle and the annuloplasty ring is desirable (FIG. 15).

Note that although in the figures shown, the two holes lay in the same plane, they could be non-planar. For example, one or both of the holes could be tilted if that resulted in better alignment. There could also be more than two locations, although due to the size of the implant, two holes are more feasible.

As in previous embodiments, the handle adapter 216 is a separate part that is assembled by inserting a distal end of the handle adapter 216 into an opening (not shown) of the ring holder 214 and is removable from the ring holder 214 by cutting a single suture. For flexible annuloplasty rings it may be desirable to keep the ring holder attached to the annuloplasty ring until all sutures are tied to ensure the desired shape is maintained. Under those circumstances, it would be helpful to be able to easily remove the handle for better access and visibility while leaving the holder attached. In an alternative embodiment, the handle adapter 216 can be made in one piece with the ring holder 214 as a monolithic part.

In other alternative embodiments, various different features from the different embodiments discussed above can also be combined in a single modified ring holder. For purposes of this description, certain aspects, advantages, and novel features of the embodiments of this disclosure are described herein. The disclosed methods, apparatus, and systems should not be construed as being limiting in any way. Instead, the present disclosure is directed toward all features and aspects of the various disclosed embodiments, alone and in various combinations and sub-combinations with one another. The methods, apparatus, and systems are not limited to any specific aspect or feature or combination thereof, nor do the disclosed embodiments require that any one or more specific advantages be present or problems be solved.

Although the operations of some of the disclosed embodiments are described in a particular, sequential order for convenient presentation, it should be understood that this manner of description encompasses rearrangement, unless a particular ordering is required by specific language set forth below. For example, operations described sequentially can in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms can vary depending on the particular implementation and are readily discernible by one of ordinary skill in the art.

In view of the many possible embodiments to which the principles of the disclosure can be applied, it should be recognized that the illustrated embodiments are only preferred examples and should not be taken as limiting the scope of the disclosure. Rather, the scope of the disclosure is defined by the following claims.

While the foregoing is a complete description of the preferred examples, various alternatives, modifications, and equivalents may be used. Moreover, it will be obvious that certain other modifications may be practiced within the scope of the appended claims.

What is claimed is:

1. An adaptable annuloplasty ring assembly with detachable handle comprising:
an annuloplasty ring configured to be secured to an annulus of a heart valve;
an annuloplasty ring holder comprising an upper proximal face, a lower distal face, and a peripheral edge portion, the annuloplasty ring holder removably attached to the annuloplasty ring such that the annuloplasty ring extends along the peripheral edge portion;
a handle with a distal attachment end;
a handle adapter extending proximally from the upper proximal face of the ring holder;
wherein the handle adapter has two attachment holes and the distal attachment end of the handle can be selectively inserted into and attached to each attachment hole, wherein the attachment holes have non-parallel axes such that the handle extends away from the upper proximal face of the ring holder at different angles when selectively attached to the two attachment holes to facilitate advancement through different surgical approaches.

2. The annuloplasty ring assembly of claim 1, wherein the distal attachment end of the handle and each of the two attachment holes are threaded for cooperative engagement.

3. The annuloplasty ring assembly of claim 2, wherein the threads between the handle and each of the two attachment holes only require about 1-2 full rotations to attach the handle.

4. The annuloplasty ring assembly of claim 1, wherein the ring holder defines an opening for receiving the handle adapter and includes several suture holes around the opening, wherein the handle adapter is removably attached to the ring holder by a single suture passed through the suture holes and over the handle adapter.

5. The annuloplasty ring assembly of claim 4, wherein the handle adapter further comprising a cutting well projecting up from the upper proximal face thereof, and wherein the single suture is arranged across the cutting well and is accessible to cutting to release the handle adapter from the ring holder.

6. The annuloplasty ring assembly of claim 1, wherein when attached to a first one of the attachment holes the handle extends away from the upper proximal face of the ring holder at a non-perpendicular angle relative to the upper proximal face of the ring holder, and when attached to a second one of the attachment holes the handle extends generally perpendicularly away from the upper proximal face of the ring holder.

7. The annuloplasty ring assembly of claim 1, wherein the ring holder further includes a crossbar extending from a first end at one side of the peripheral edge portion to a second end at an opposite side of the peripheral edge portion, and between the peripheral edge portion and the crossbar the ring holder provides a pair of relatively large visibility windows that together occupy a large cross-sectional area within the peripheral edge portion, and wherein the handle adapter extends proximally from the crossbar and the axes of the two holes lie in a common plane aligned with the crossbar.

8. The annuloplasty ring assembly of claim 7, wherein a width of the crossbar at one end is narrow, wherein the width of the crossbar at the opposite end is wide and defines an opening for receiving the handle adapter.

9. The annuloplasty ring assembly of claim 8, wherein the annuloplasty ring holder removably attaches to the annuloplasty ring using at least one suture.

10. The annuloplasty ring assembly of claim 7, further including a cutting well projecting upward from the proximal face of the ring holder at the narrow end of the crossbar over which the at least one suture is suspended, the cutting well providing a space for a suture cutting instrument to sever the at least one suture and detach the annuloplasty ring holder from the annuloplasty ring.

11. An adaptable annuloplasty ring assembly with detachable handle comprising:
an annuloplasty ring configured to be secured to an annulus of a heart valve;
an annuloplasty ring holder comprising an upper proximal face, a lower distal face, and a peripheral edge portion and a crossbar extending from a first end at one side of the peripheral edge portion to a second end at an opposite side of the peripheral edge portion, and between the peripheral edge portion and the crossbar the ring holder provides a pair of relatively large visibility windows that together occupy a large cross-sectional area within the peripheral edge portion, the annuloplasty ring holder removably attached to the annuloplasty ring such that the annuloplasty ring extends along the peripheral edge portion;
a handle with a distal attachment end;
wherein the ring holder defines two attachment holes opening proximally and the distal attachment end of the handle can be selectively inserted into and attached to each attachment hole, wherein the attachment holes have non-parallel axes that lie in a common plane aligned with the crossbar such that the handle extends away from the upper proximal face of the ring holder at different angles when selectively attached to the two attachment holes to facilitate advancement through different surgical approaches.

12. The annuloplasty ring assembly of claim 11, wherein when attached to a first one of the attachment holes the handle extends away from the upper proximal face of the ring holder at a non-perpendicular angle relative to the upper proximal face of the ring holder, and when attached to a second one of the attachment holes the handle extends generally perpendicularly away from the upper proximal face of the ring holder.

13. The annuloplasty ring assembly of claim 11, wherein the distal attachment end of the handle and each of the two attachment holes are threaded for cooperative engagement.

14. The annuloplasty ring assembly of claim 13, wherein the threads between the handle and each of the two attachment holes only require about 1-2 full rotations to attach the handle.

15. The annuloplasty ring assembly of claim 11, wherein the ring holder has a handle adapter removably attached to the upper proximal face thereof that defines the two attachment holes.

16. The annuloplasty ring assembly of claim 15, wherein the ring holder defines an opening for receiving the handle adapter and includes several suture holes around the opening, wherein the handle adapter is removably attached to the ring holder by a single suture passed through the suture holes and over the handle adapter.

17. The annuloplasty ring assembly of claim 16, wherein the handle adapter further comprising a cutting well projecting up from the upper proximal face thereof, and wherein the single suture is arranged across the cutting well and is accessible to cutting to release the handle adapter from the ring holder.

18. The annuloplasty ring assembly of claim 15, wherein the handle adapter extends proximally from the crossbar and the axes of the two holes lie in a common plane aligned with the crossbar.

19. The annuloplasty ring assembly of claim 18, wherein a width of the crossbar at one end is narrow, wherein the width of the crossbar at the opposite end is wide and defines an opening for receiving the handle adapter.

20. The annuloplasty ring assembly of claim 19, wherein the annuloplasty ring holder removably attaches to the annuloplasty ring using at least one suture, and further including a cutting well projecting upward from the proximal face of the ring holder at the narrow end of the crossbar over which the at least one suture is suspended, the cutting well providing a space for a suture cutting instrument to sever the at least one suture and detach the annuloplasty ring holder from the annuloplasty ring.
